(19) United States
(12) Patent Application Publication
(10) Pub. No.: US 2025/0108460 A1
(43) Pub. Date: Apr. 3, 2025

(54) LASER PATTERNING APPARATUS OF A PROCESSING OBJECT AND A METHOD THEREOF AND A THREE-DIMENSIONAL PROCESSING OBJECT PROCESSED THEREBY

(71) Applicant: Byoung Chan CHOI, Gwangmyeong-si, Gyeonggi-do (KR)

(72) Inventor: Byoung Chan CHOI, Gwangmyeong-si, Gyeonggi-do (KR)

(21) Appl. No.: 18/725,803

(22) PCT Filed: Dec. 29, 2021
(86) PCT No.: PCT/KR2021/020115

§ 371 Date: Jun. 30, 2024

(51) Int. Cl. B23K 26/70; B23K 26/03; B23K 31/12

(52) U.S. Cl. CPC B23K 26/70; B23K 26/032; B23K 31/12

(57) ABSTRACT

The present invention relates to a method of laser patterning of a processing object, wherein, after loading a three-dimensional processing object into a laser patterning apparatus, a predetermined z-axis processing height is primarily set, and processing is performed after matching the two-dimensional position information which has been generated by two-dimensional measuring the shape of the processing object with the three-dimensional processing pattern data provided for laser patterning processing of the processing object, and the z-axis processing height is secondarily set until the z-axis processing height, which is based on the three-dimensional pattern processing pattern data through the processing shape analysis of the patterned processing object, reaches a preset target value and then processing is performed. Which provides the advantage of not requiring a separate 3D profiling process.

### TECHNICAL FIELDS

The present invention relates to a laser patterning apparatus of a processing object and a method thereof and a three-dimensional processing object processed thereby.

### BACKGROUND ART

Laser processing refers to processing an object using a laser beam, and recently, laser processing may be used to form a predetermined pattern on the surface of the object to be processed. The laser patterning apparatus used in such laser processing is a apparatus that forms a predetermined pattern on the object to be processed using a laser.

However, conventional laser patterning apparatus have not been able to perform patterning on a curved three-dimensional object. For example, in the case of patterning a three-dimensional curved surface of a living such as an intraocular lens and a smart lens, it has been difficult to use because it has not been able to secure a high degree of precision and dimensional control.

### SUMMARY OF INVENTION

### Technical Problem

Embodiments of the present invention are directed to providing a laser patterning apparatus, which affects the coupling force and adhesion force of attached parts, and a method thereof, and a three-dimensional object processed thereby. In addition, embodiments of the present invention are directed to providing a laser patterning apparatus, which can produce a pattern of a micro-size to that of a nano-size using a pulsed laser beam, and a method thereof, and a three-dimensional object processed thereby.

In addition, embodiments of the present invention are directed to providing a laser patterning apparatus, which can uniformly process a line width of a nano-size to that of a micro-size through a dynamic focusing module that can adjust the focus height of a laser beam, and a method thereof, and a three-dimensional object processed thereby.

In addition, embodiments of the present invention are directed to providing a laser patterning apparatus, which does not require a separate three-dimensional profiling process since for processing the object to be processed, a three-dimensional pattern is input after being loaded, two-dimensional position information is generated, matched, processed, and analyzed, and then the z-axis processing height is set through the dynamic focusing module to complete patterning processing, and a method thereof, and a three-dimensional object processed thereby.

The technical problems of the present invention are not limited to the technical problems mentioned above, and other technical problems not mentioned may be clearly understood by those skilled in the art from the following descriptions.

### Solution to Problem

In one embodiment of the laser patterning method of the processing object according to the present invention, a three-dimensional processing object is loaded into the laser patterning apparatus, then a pre-determined z-axis processing height is primarily set, and the two-dimensional position information, which has been generated by measuring the shape of the processing object in two dimensions, is matched with the three-dimensional processing pattern data provided for the laser patterning processing of the processing object, and then processing is performed. When the processing pattern position (x, y, z) of the processing object based on the three-dimensional pattern processing pattern data is mismatched or the dimension (width, depth) and spacing of the processing pattern are uneven through the processing shape analysis of the pattern processing object by the processing, the z-axis processing height can be secondarily set until the processing pattern position, the pattern processing width, or the spacing reaches a preset target value.

Here, the two-dimensional position information may be acquired from the two-dimensional inspection of the processing object using a camera equipped with CCD or CMOS.

In addition, the matching of the two-dimensional position information with the three-dimensional processing pattern data can be performed after calculating center point information through the outer contour recognition or area calculation of the processing object acquired through the generation of the two-dimensional position information.

In addition, the matching of the two-dimensional position information and the three-dimensional processing pattern data can be performed after calculating center position, tilt information, and the like of the processing object through the outer contour recognition or area calculation of the processing object acquired through the generation of the two-dimensional position information.

In addition, object based on the height information of the design drawing or the actual measured height information of the processing object, the pre-determined z-axis processing height can be determined by any one of the z-axis according to the processing stage on which the processing object is loaded and the z-axis according to the beam regulator of the laser patterning apparatus patterning the processed.

In addition, the processing shape analysis of the processing object can be performed by measuring any one of the processing widths and the spacing of the pattern processed to the processing object through the camera equipped with CCD or CMOS.

In addition, the processing shape analysis of the processing object can be performed by measuring any one of the processing widths and the spacing of the pattern processed to the processing object using one of an optical coherence tomography (OCT), a laser interferometer, a confocal microscope, and a two-photon microscope.

In one embodiment of the laser patterning method of the processing object according to the present invention, a three-dimensional processing object loading step of loading a three-dimensional processing object, which is a three-dimensional processing object, into the laser patterning apparatus, a three-dimensional processing pattern input step of inputting three-dimensional processing pattern data for the laser patterning processing of the processing object, a two-dimensional position information generation step of generating two-dimensional position information by measuring the shape of the processing object in two dimensions, a z-axis primarily setting step of setting a pre-determined z-axis processing height, which is the processing height of the processing object to be processed through the laser patterning apparatus, and a pattern and position information matching step of matching the two-dimensional position information with the three-dimensional processing pattern data.

Here, after the pattern and position information matching step, the processing step of performing processing at the z-axis processing height set in the primary setting step can be further included.

In addition, the processing step can include a z-axis secondary setting step that analyzes the object to be processed by the z-axis primary setting step in a two-dimensional or three-dimensional manner and then repeats processing by assigning a new z-axis processing height until the z-axis processing height is reached.

In addition, the z-axis secondary setting step can include:
a processing shape analysis process that two-dimensionally analyzes the pattern processing shape of the processing object using a camera equipped with a CCD or CMOS, and a z-axis correction process that assigns a new z-axis processing height when one of the pattern processing width, pattern spacing, and pattern depth of the processing object analyzed by the processing shape analysis process does not reach one of the pattern width, pattern spacing, and pattern depth on the three-dimensional processing pattern data.

Alternatively, the z-axis secondary setting step can include a processing shape analysis process that two-dimensionally or three-dimensionally analyzes the pattern processing width or pattern spacing of the processing object using one of an optical coherence tomography (OCT), a laser interferometer, a confocal microscope, or a two-photon microscope, and a z-axis correction process that assigns a new z-axis processing height when one of the pattern processing width, pattern spacing, and pattern depth of the processing object analyzed by the processing shape analysis process does not reach one of the pattern width, pattern spacing, and pattern depth on the three-dimensional processing pattern data.

In addition, the processing shape analysis process can be performed by measuring any one of the processed line widths and the spacing of the pattern processed on the surface of the object to be processed.

In addition, the method may further include a quality analysis and reporting step of analyzing and reporting any one of a shape of a pattern, a width of a pattern, a depth of a pattern, a spacing between patterns, a wavelength and an output of a laser beam, a pulse width, a shape of a beam, a scanning speed, and a spot size for the processing object to which the pattern is processed.

The laser patterning apparatus of the processing object according to an embodiment of the present invention includes a laser generator, a beam conversion apparatus configured to adjust the size or shape of the laser beam generated by the laser generator, a beam adjuster configured to include a dynamic focusing module to adjust the z-axis focus position of the laser beam via the beam conversion apparatus and a scan head to adjust the x-axis and y-axis focus positions of the laser beam, and a controller configured to control the beam adjuster to enable laser patterning to the three-dimensional processing object, wherein a three-dimensional processing object is loaded into the laser patterning apparatus, then a pre-determined z-axis processing height is primarily set, and the two-dimensional position information, which has been generated by measuring the shape of the processing object in two dimensions, is matched with the three-dimensional processing pattern data provided for the laser patterning processing of the processing object, and then the controller performs processing and controls the beam adjuster for the z-axis processing height to be secondarily set until the z-axis processing height, which is based on the three-dimensional pattern processing pattern data, is reached through the processing shape analysis of the pattern processing object by the processing.

In addition, the present invention provides a three-dimensional processing object that is patterning processed with any one of the laser patterning method and the laser patterning apparatus.

### Advantageous Effects of Invention

According to an embodiment of the laser patterning apparatus and method of the processing object and the three-dimensional processing object processed by the same according to the present invention, the following various effects can be achieved.

First, since the surface of the processing object can be patterned to have a precision consistent with the standards of the attached parts, the performance and reliability of the processing object can be improved.

Second, a pattern of micro-size to that of nano-size can be produced using a pulsed laser beam.

Third, a line width of nano to that of micro size can be uniformly processed on the three-dimensional surface through a dynamic focusing module that can adjust the focus height of the laser beam.

Fourth, completed processing can be performed without using separate surface information of the three-dimensional object for the three-dimensional patterning processing on the surface of the processing object.

### BRIEF DESCRIPTION OF DRAWINGS

FIG. 1 is a diagram showing a path of a laser beam of a laser patterning apparatus of a processing object according to an embodiment of the present invention.

FIGS. 2 and 3 are diagrams showing a beam path of a laser patterning of a three-dimensional processing object according to an embodiment of the present invention.

FIGS. 4 and 5 are flowcharts for explaining a flow of processing a processing object according to an embodiment of the present invention.

FIG. 6 is a diagram showing an exemplary three-dimensional processing object according to an embodiment of the present invention, and FIG. 7 is an enlarged cross-sectional view taken along line A-A of FIG. 6.

FIGS. 8 and 9 are diagrams showing a pattern shape according to an embodiment of the present invention.

FIG. 10 is a diagram showing an intraocular lens where a patterning has completed when a three-dimensional processing object is an intraocular lens according to an embodiment of the present invention.

### DESCRIPTION OF EMBODIMENTS

Hereinafter, specific embodiments of the present invention are disclosed with references. Disclosures including drawings are exemplary embodiments, and the present invention is not limited thereto.

In the description of the present invention, a detailed description of known technologies related to the present invention are omitted when it is determined that they may unnecessarily obscure the gist of the present invention. The terms to be described later are terms defined in consideration of the functions in the present invention, and they may vary depending on the intention or customs of the user or the operator. Therefore, the definition should be made based on the contents throughout this specification.

The technical ideas of the present invention is determined by the claims, and the following embodiments are merely means for efficiently explaining the technical ideas of the present invention to those skilled in the art to which the present invention pertains.

The laser patterning apparatus for the processing object according to the present invention may be used to process an implant for the living body (living implant), for example, intraocular lenses, dental implants, orthopedic implants, etc. Meanwhile, at least one of a micro pattern and a nano pattern that affects cell alignment and movement direction in a living implant may be patterned, including the laser generation unit and the beam adjustment unit. Here, as an example of the subject to be processed, a subject that may be implanted or implanted in a living body is given, but it is not limited thereto, and it is of course also possible to include a subject to be processed that includes a pattern that may be used within or with the human body. The object to be processed that includes a pattern that may be used within or with the human body is a concept that includes a smart lens s recently launched.

Furthermore, it may be used to process electrical and electronic components, optical components, automobile components, and aerospace components.

Hereinafter, the laser patterning apparatus and method for such object to be processed are separately described, however, the specific steps of the method omitted in the description of the apparatus may be performed in the apparatus according to the embodiment of the present invention.

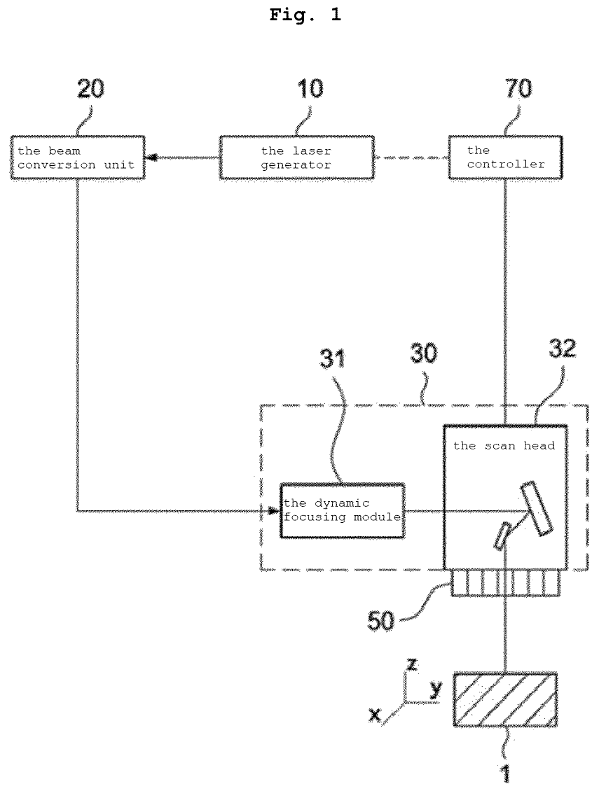

FIG. 1 is a diagram illustrating a path of a laser beam of a laser patterning apparatus for a subject to be processed according to an embodiment of the present invention.

Referring to FIG. 1, the laser patterning apparatus according to an embodiment of the present invention may include a laser generation unit 10, a beam conversion unit 20, a beam adjustment unit 30, a condensing unit 50, a z-axis corrector (not shown), and a control unit 70 to perform fine patterning on a sample 1, which is a three-dimensional subject.

The laser generation unit 10 may generate a laser beam for patterning. Specifically, the laser generation unit 10 may use a pulsed laser source. Therefore, the laser generation unit 10 may generate a laser beam from nanosecond, picosecond, femtosecond, and attosecond. Among these, the laser beam may be a microwave laser with a duration time of 1 to 1000 femtosecond. Specifically, the laser generation unit 10 may generate a pulsed laser beam with a duration time within the femtosecond range. Here, the pulse repetition rate may be in the range of two digits kHz to maximum three digits kHz or in the range of MHz. The wavelength of the laser beam can use any of the laser wavelengths located in the ultraviolet region from the infrared region. For example, it may include ultraviolet wavelengths, green wavelengths, and infrared wavelengths.

The laser patterning apparatus of the processing object according to an embodiment of the present invention may pattern a pattern having a width, a gap, and a depth of a nano unit in a micro unit in the sample 1, which is a three-dimensional processing object. Various pattern sizes can be realized by converting and using laser wavelengths according to the designed pattern size and material type. For example, when the three-dimensional processing object is an intraocular lens which is one of living implants, a laser beam may be irradiated at once to the entire surface of the processing object having a diameter of 10 mm or more (preferably, 12 mm) to generate a pattern of nano units in a few micro units. In addition, the laser patterning apparatus of the processing object according to an embodiment of the present invention can overcome the diffraction limit of the condensing unit and realize a nano-scale pattern by converting the wavelength of the laser to have a short wavelength of the ultraviolet region according to the material type.

The laser beam generated by the laser generation unit 10 may be a pulsed femtosecond laser beam, and may pass through the beam conversion unit 20 and the beam adjustment unit 30.

The beam conversion unit 20 may adjust the size or shape of the laser beam generated by the laser generation unit 10. Specifically, the beam conversion unit 20 may expand or contract the laser beam. In addition, the beam conversion unit 20 may generate the laser beam as a collimated beam with small dispersion or concentration. As a result, the laser beam generated by the laser generation 10 may be a collimated beam being expanded or contracted via the beam conversion unit 20.

The size of the laser beam changed by the beam conversion unit 20 may be the size of the laser beam incident on the last stage of the laser patterning apparatus of the processing object according to an embodiment of the present invention. The beam conversion unit 20 may change the diameter of the laser beam generated by the laser generation unit 10 and output the changed laser beam. The beam conversion unit 20 may be adjustable manually or automatically. Here, the beam conversion unit 20 can convert the shape of the Gaussian beam into a flattop beam or a multi-spot shape and output it. The shape of the flattop beam may be one of circular, polygonal, and ring shapes.

In addition, various optical elements such as a beam attenuator, a polarizing plate, a half-wave plate, a splitter, a filter, and a shutter may be further included.

The beam adjustment unit 30 may adjust the focus height and focus position of the laser that may be irradiated to the sample 1, which is a three-dimensional processing object. The beam adjustment unit 30 may include a dynamic focusing module 31 and a scan head 32. The dynamic focusing module 31 of the beam adjusting unit 30 may adjust the focus height of the laser beam, and the scan head 32 of the beam adjusting unit 30 may adjust the focus position of the laser beam along the sample 1, which is a three-dimensional processing object.

The dynamic focusing module 21 may adjust the focal position of the laser beam passing through the condensing unit 50 according to the set z-axis processing height through matching the z-axis height of the sample 1, which is a three-dimensional processing object to be described later, with the two-dimensional position information by the z-axis primary setting step. Specifically, the dynamic focusing module 31 may include two or more lenses (not shown). The focus of the laser beam passing through the condensing unit 50 may be adjusted by adjusting the divergence and convergence of the laser beam passing through the dynamic focusing module 31 by adjusting the interval between the respective lenses. In addition, the dynamic focusing module 31 may be configured with a reflective optical system.

The dynamic focusing module 31 may adjust the focus height of the laser beam via the beam adjusting unit 30, that is, the z-axis position of the focus. The dynamic focusing module 31 may adjust the z-axis position of the laser beam, that is, the focus height of the laser beam, by adjusting the convergence and divergence of the laser beam via the beam conversion unit 20.

The dynamic focusing module 31 may adjust and irradiate the focal length of the laser beam transmitted to the scan head 32 by driving a motor (not shown) that performs horizontal reciprocal movement. For example, when one lens inside the dynamic focusing module 31 moves to the right, the focus of the laser beam is moved away from the sample 1, which is a three-dimensional processing object, and thus the laser beam may be moved from the z-axis to the upper side of the paper surface of FIG. 1. Therefore, the focus height of the laser beam may be shortened. On the contrary, when one lens inside the dynamic focusing module 31 moves to the left, the laser beam is closer to the sample 1, which is a three-dimensional processing object, and thus the focus of the laser beam may be moved from the z-axis to the lower side of the paper surface of FIG. 1. Therefore, the focus height of the laser beam may be longer. Therefore, the focus position of the laser beam incident on the sample 1, which is a three-dimensional processing object, may be controlled in the z-axis direction.

The dynamic focusing module 31 may pattern along the height of the surface of the three-dimensional shape of the sample 1, which is a three-dimensional processing object. For example, since the intraocular lens, which is one of the living implants, has a curved shape, the height (i.e., the z-axis) of the position at which the pattern needs to be patterned by the laser beam may be different from each of the x-axis and the y-axis. The adjustment of the position on the z-axis of the focus of the laser beam through the dynamic focusing module 31 may uniformly pattern corresponding to the different z-axis positions for each x-axis and y-axis coordinates. In addition, the pattern may be patterned from a nanoscale to a microscale width. In addition, the shape of the pattern may be a point, a dotted line, a line, a poly line, a circle, a polygon, or the like. The dynamic focusing module 31 may move the internal optical system or each of the lenses included in the optical system. Therefore, the height of the laser beam may be highly controlled in real time, and thus the uniformity of the line width at the surface of the sample 1, which is a three-dimensional processing object, may be improved, and productivity may be improved.

The x-axis and the y-axis focus positions of the laser beam adjusted by the dynamic focusing module 31 may be adjusted by the scan head 32.

The scan head 32 may adjust the x-axis and the y-axis focus positions of the sample 1, which is a a three-dimensional processing object. In other words, the scan head 32 includes an x-axis scan mirror (not shown) and a y-axis scan mirror (not shown) although not shown in the drawing, and can perform two-dimensional scanning. The laser beam having the z-axis focus position adjusted by the dynamic focusing module 31 may be finely controlled in the x-axis and y-axis directions along the curved surface of the sample 1, which is a three-dimensional processing object, through the x-axis scan mirror and the y-axis scan mirror.

The x-axis scan mirror and the y-axis scan mirror of the scan head 32 may reflect the laser beam in the direction for patterning to irradiate the laser beam to a desired position of the sample 1, which is a three-dimensional processing object. The x-axis scan mirror and the y-axis scan mirror are configured with a pair of scan mirrors by galvanometer type, and the pair of scan mirrors may deflect the laser beam in one of the axes transverse to the x-y plane, respectively.

Therefore, as described above, the beam adjustment unit 30 may adjust the focus height and the focus position of the laser beam. The laser beam may be expanded or contracted while passing through the beam conversion unit 20 to be adjusted in size, and may be generated as a collimated beam to be refracted in a controlled direction. The laser beam passing through the beam conversion unit 20 may be adjusted in the z-axis focus position by the dynamic focusing module 31, and the coordinates of x, y may be adjusted by the scan head 32 to adjust the focus position of the laser beam to correspond to the sample 1, which is a three-dimensional processing object.

A condensing unit 50 for focusing the femtosecond laser beam passing through the dynamic focusing module 31 and the scan head 32 to the sample 1, which is a three-dimensional processing object, may be disposed under the beam adjustment unit.

The condensing unit 50 may condense the laser beam. The condensing unit 50 may condense the laser beam passing through the beam adjustment unit 30 to irradiate the laser beam to the sample 1, which is a three-dimensional processing object. The condensing unit 50 may include a telecentric F-theta lens or an F-theta lens. Therefore, a micro or nano-scale fine pattern may be processed.

Through these configurations, at least one or more of various parameters such as the irradiation position, the focal length, the pulse waveform of the output laser beam, the irradiation time, the scanning speed, the divergence and convergence characteristics, the shape of the beam, and the pattern shape may be adjusted.

The control unit 70 may input the designed three-dimensional processed pattern data to perform patterning on the surface of the sample 1, which is a three-dimensional processing object having a curved surface, and extract the focus position data of the x-axis, y-axis, and z-axis. Based on this data, the scan head 32 may control the two-dimensional focus position data of the x-axis and y-axis. In addition, the dynamic focusing module 31 may control the focus position data of the z-axis to control the three-dimensional processed pattern data in real time. Therefore, a fine pattern, having widths and depth of a micro-scale pattern to a nano-scale pattern, may be produced on the surface of the processing object.

The laser patterning apparatus of the processing object according to an embodiment of the present invention may further include an ultrafine stage (not illustrated). When the sample 1, which is a three-dimensional processing object, is mounted to the stage for processing, it is likely to deform out of the effective focal distance of the optical system. Therefore, it is possible to control the sample 1, which is a three-dimensional processing object, to be located within an effective focal length and an effective processing area of the dynamic focusing module 31 and the scan head 32 according to a combination of a large number of axes in a coordinate system defined through a nano-scale ultrafine stage. The “z-axis processing height” described below may be determined by at least one of a mechanical z axis or an optical z axis, and the mechanical z axis may be defined as a change in the height of the sample 1, which is a three-dimensional processing object, by the above-described ultrafine stage, and the optical z axis may be defined as a change in the focus of the laser beam to the sample 1, which is a three-dimensional processing object, by the dynamic focusing module 31 among the configurations of the beam adjusting unit 30.

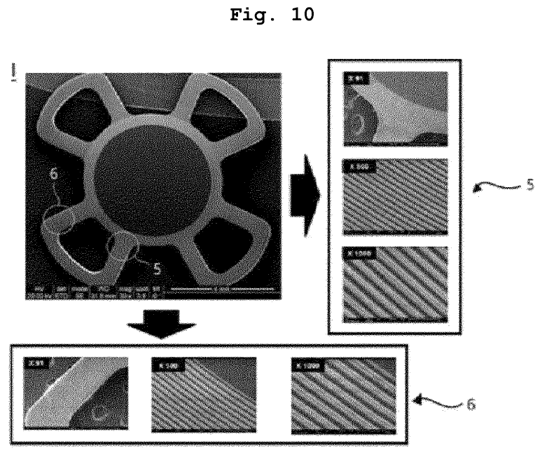

Meanwhile, as illustrated in FIG. 10, the three-dimensional processing object may be an intraocular lens. However, the intraocular lens is merely exemplary of the three-dimensional processing object, and it is of course, it may include any object that can be implanted or inserted into the human body, such as an implant, a stent, and an object that can be used together with the human body. Furthermore, it is of course, it may include an electrical and electronic component, an optical component, a car component, and an aerospace component.

As illustrated in FIG. 10, the intraocular lens may include an optic portion in the central region and a haptic portion in the outer region. The laser patterning apparatus of the processing object according to an embodiment of the present invention may irradiate a femtosecond laser beam to the haptic portion of the intraocular lens to perform fine patterning. By forming various shapes of micro or nano scale patterns in the haptic portion, the cells may be aligned with directionality and movable and adhere.

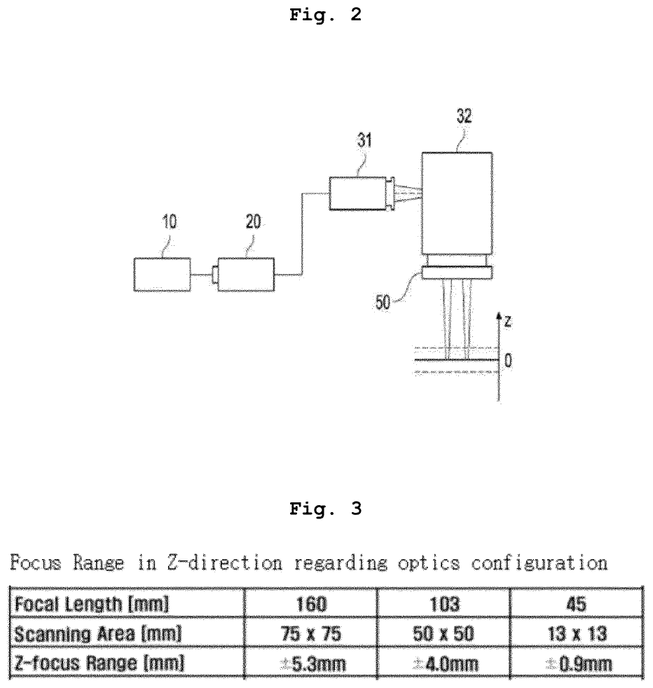

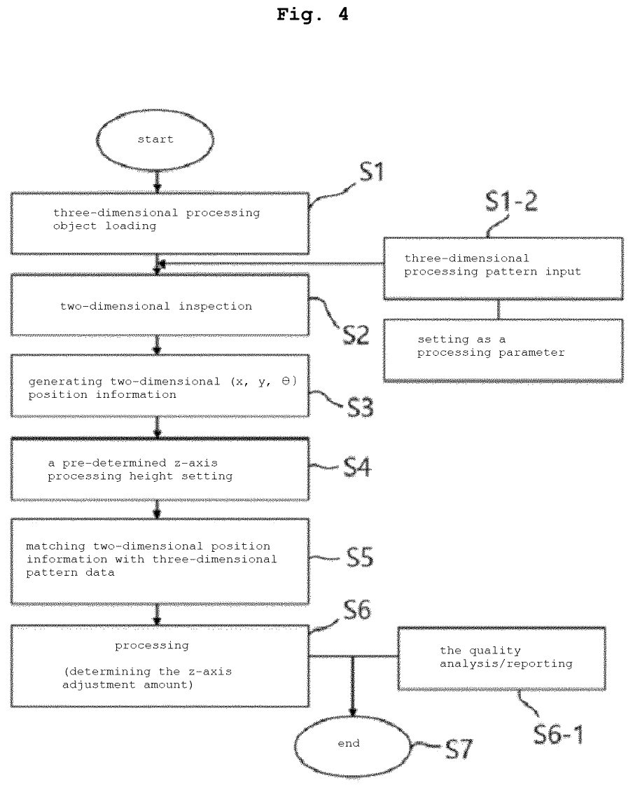

FIGS. 2 and 3 are drawings illustrating a beam path of laser patterning of the three-dimensional processing object according to an embodiment of the present invention.

Referring to FIG. 2, the laser beam generated in the laser generation 10 passes through the beam conversion apparatus 20 and is transmitted to the beam adjusting unit 30. The z-axis of the laser beam transmitted to the beam adjusting unit 30 may be adjusted by the dynamic focusing module 31 and the x-axis and the y-axis of the laser beam transmitted to the beam adjusting unit 30 may be adjusted by the scan head 32.

Referring to FIG. 3, the laser beam having the x, y, and z-axis adjusted by the dynamic focusing module 31 and the scan head 32 may be irradiated to the surface of the sample 1, which is a three-dimensional processing object.

For example, the size of the laser beam irradiated to the x-axis and y-axis fields (i.e., imaging surfaces) having different heights of the sample 1, which is a three-dimensional processing object, may be the same. The size of the scannable x-axis and y-axis may be determined according to the specification of the focusing lens of the condensing unit 50. When the focal length of the condensing unit 50 is 160 mm and the scanning range of the x-axis and the y-axis is 75 mm×75 mm, the focal range of the z-axis may be ±5.3 mm. In addition, when the focal length is 103 mm, and the scanning range of the x-axis and the y-axis is 50 mm×50 mm, the focal range of the z-axis may be ±4.0 mm, and when the focal length is 45 mm, and the scanning range of the x-axis and the y-axis is 13 mm×13 mm, the focal range of the z-axis may be ±0.9 mm. That is, according to the 3D pattern data of the 3D processing object input to the controller, the specifications of the beam adjusting unit 30 and the condensing unit 50 may be differently configured to determine the patterning area and height of the processing object. In addition, the dynamic focusing module 31 may adjust the z-axis height corresponding to the x-axis and y-axis coordinate values adjusted by the scan head 32.

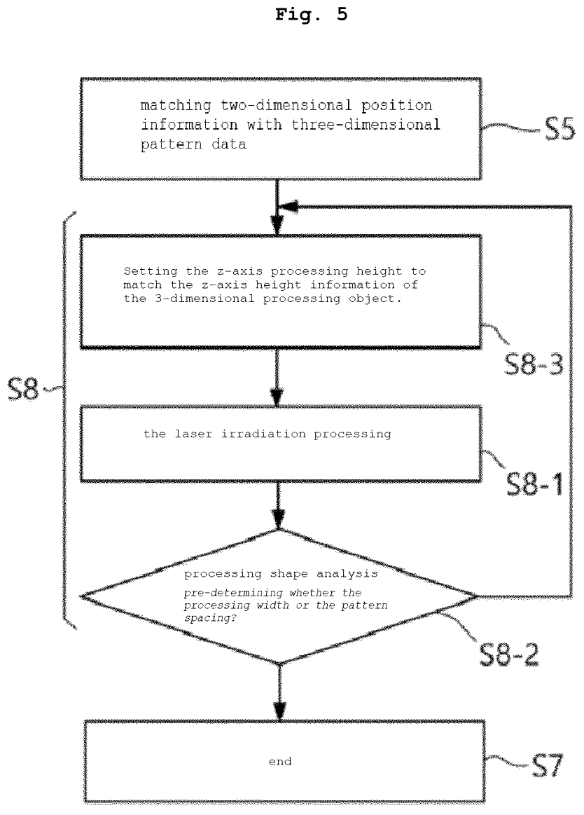

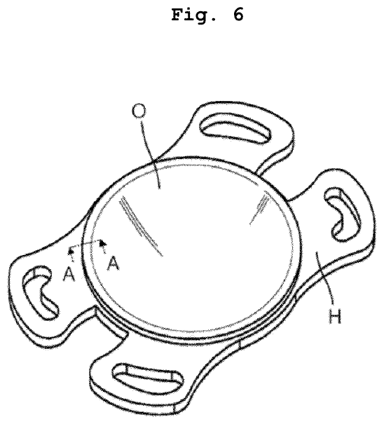

FIGS. 4 and 5 are flowcharts for explaining the flow of processing a processing object according to an embodiment of the present invention.

Referring to FIG. 4, a method of processing a three-dimensional processing object is performed by loading a sample 1, which is a three-dimensional processing object (hereinafter, referred to as a three-dimensional processing object loading step S1), performing a two-dimensional inspection of the loaded three-dimensional processing object (hereinafter, referred to as a two-dimensional inspection step S2), generating and inputting two-dimensional position information (hereinafter, referred to as a two-dimensional position information generation step S3), setting and inputting a pre-determined z-axis processing height (hereinafter, referred to as a z-axis primary setting step S4), matching two-dimensional position information with three-dimensional pattern data (hereinafter, referred to as a pattern and position information matching step S5), irradiating a laser beam and processing (hereinafter, referred to as a processing step S6), and completing laser patterning (S7) to a sample 1, which is the final three-dimensional processing object (hereinafter, referred to as a three-dimensional processing object S1).

Between the three-dimensional processing object loading step S1 and the two-dimensional inspection step S2, the three-dimensional processing pattern input step S1-2 provided to pattern the sample 1, which is the three-dimensional processing object, may be further included, and before the completing step S7 after the processing step S6, the quality analysis/reporting step s6-1 may be further included. In addition, the z-axis primary setting step S4 is explained only as being performed after the two-dimensional position information generation step S3 in the laser patterning method of the processing object according to an embodiment of the present invention, though, depending on the embodiment, it may be performed immediately after the three-dimensional processing object loading step S1. In other words, it is sufficient for the z-axis primary setting step S4 to be performed at least before the pattern and position information matching step S5.

In more detail, the sample 1, which is a processing object provided as a transparent body, is loaded into a processing stage such as an ultrafine stage (s1).

When the loading of the sample 1, which is a three-dimensional processing object, is completed on the processing stage, the two-dimensional inspection is performed (S2). The two-dimensional inspection herein may mean the external inspection of the sample 1, which is a three-dimensional processing object.

Here, a three-dimensional processing pattern input step S1-2 provided to pattern the sample 1, which is a three-dimensional processing object, may be further included between the three-dimensional processing object loading step S1 and the two-dimensional inspection step S2. The three-dimensional processing pattern input step S1-2 may be an input step of a three-dimensional design diagram in which a pattern to be laser processed on the surface of the sample 1, which is a three-dimensional processing object, is set as a processing parameter.

After a 2D inspection step S2 is performed, a 2D position information generation step S3 may be performed. 2D position information refers to 2D coordinate information about a specific point on the x-y, x-z, and y-z planes. The two-dimensional coordinates for a specific point on the x-y plane can be represented by the (x, y) variable, the two-dimensional coordinates for a specific point on the x-z plane can be represented by the (x, θ) variable, and the two-dimensional coordinates for a specific point on the y-z plane can be represented by the (y, θ) variable. Therefore, the two-dimensional position information may include the position information of a line and a plane in the vertical direction and the horizontal direction. In other words, the final 3D processing object manufactured according to the 2D shape information included in the 2D position information may coincide with the position information.

Here, the two-dimensional shape information may be acquired from the sample 1, which is a three-dimensional processing object, using a camera (not shown) equipped with CCD and CMOS. The two-dimensional shape information may provide information about the alignment state (tilting of the processing stage to be described later) of the sample 1, which is a three-dimensional processing object, and the center point (x-y coordinates). In particular, the center point information may be acquired through the recognition of the outline or area calculation of the sample 1, which is a three-dimensional processing object. In addition, the center position, contraction, expansion, and tilting state of the sample 1, which is a three-dimensional processing object, may be identified through the area calculation, and this information may be used in the determining of the z-axis adjustment amount in the pattern and position information matching step S6 or the z-axis secondary setting step S8 to be described later. This will be described in more detail later.

After performing the two-dimensional position information generation step S3, the z-axis primary setting step S4 may be performed. It has already been described that the performing of the z-axis primary setting step S4 may be performed directly after the loading step S1 of the three-dimensional processing object, if it is at least performed only before the pattern and position information matching step S5.

Here, the z-axis primary setting step S4 may be a step of inputting the primary information about the position of the pattern (z-axis processing height) processed on the outer surface of the sample 1, which is a three-dimensional processing object, using the laser patterning apparatus of the processing object according to an embodiment of the present invention. However, the z-axis processing height set and input in the z-axis primary setting step S4 does not mean the processing height of the pattern patterned on the final three-dimensional processing object, and may have the meaning as an input step for matching with the z-axis processing height information of the sample 1, which is a three-dimensional processing object, in the pattern and position information matching step S5 to be described later.

When the performing of the z-axis primary setting step S4 is completed, the pattern and position information matching step S6 may be performed to mutually match the three-dimensional processing pattern data input in the previously performed three-dimensional processing pattern input step S1-2 with the two-dimensional position information generated in the two-dimensional position information generation step S3.

The pattern and position information matching step S6 may inspect the position and alignment state of the sample 1, which is a three-dimensional processing object, already loaded. The inspection is a procedure of confirming the position information included in the two-dimensional position information to be matched, and the sample 1, which is a tested three-dimensional processing object, may match the previously input position information with the position information of the sample 1, which is a three-dimensional processing object. That is, the loaded processing object or the scan head 32 may be moved to be aligned so that the inspected position information of the sample 1, which is a three-dimensional processing object, and the position information included in the two-dimensional position information can overlap.

Referring to FIG. 4, the alignment state may be achieved through the matching of the three-dimensional pattern data A and the two-dimensional (x, y, θ) position information. As already described, the two-dimensional position information refers also to two-dimensional coordinate information about a specific point on the x-y plane, x-z plane, and y-z plane, and the alignment of the sample 1, which is actually loaded, may be controlled. In order to confirm the alignment state by the matching (c) of each data and correct the alignment state, the loaded processing stage (not shown) of the sample 1 may be rotated or tilted, and may be moved from the position before the alignment to be aligned.

When the performing of the pattern and position information matching step S5 is completed, the processing step S6 to be processed by the laser according to the input 3D processing pattern data may be performed on the surface of the sample 1, which is the aligned 3D processing object.

Here, the processing step S6 may be based on the input 3D processing pattern data A, focus position 2D data of x-axis and y-axis may be the data of controlling the scan head 32 among the configurations of the beam adjusting unit 30, and the processing height setting data of the z-axis input by the above-described z-axis primary setting step S4 may be the data of controlling the dynamic focusing module 31 among the configurations of the beam adjusting unit 30.

More specifically, the processing step S6 may be defined as the step of performing the laser processing on the sample 1, which is the position-aligned processing object. Here, the meaning of the laser processing means that the sample 1, which is the processing object, is processed by the laser according to the information of the pattern. In this case, the controller may control the apparatus with the laser processing parameter information such as the wavelength, output, pulse width, the shape of the beam, the spot size, and the scanning speed of the laser, and the information of the width of the pattern and the interval between the patterns, so that the laser may be irradiated.

After the execution of the processing step S6, the quality analysis and reporting step S6-1 of the patterned 3D processing object may be performed selectively. The quality analysis and reporting step S6-1 may be a step of examining whether the size of the pattern, the pattern interval, the pattern depth, and the surface roughness of the processed surface processed by the laser, correspond to a predetermined criterion. The step may include the examining whether the pattern is formed to the 3D processing object by the laser in accordance with the position information included in the 2D position information data. Of course, the accordance degree of the pattern may be predetermined by one of ordinary skill in the art, and for example, the width between the concave-convex portions formed by the pattern processing, the depth of the concave-convex portions, and the width may be a criterion for determining the accordance degree. Meanwhile, the quality analysis and reporting step S6-1 includes storing information related to the quality while performing the quality analysis, and further outputting and reporting the stored information. The uniform patterning quality of the sample 1, which is the processing object, may be maintained by accumulating the quality analysis information.

The quality analysis and reporting step S6-1 may be performed by a non-contact method by a camera provided as CCD and CMOS. In addition, one of an optical coherence tomography (OCT), a laser interferometer, a confocal microscope, or a two-photon microscope may be used. When the quality analysis and reporting step S6-1 is completed, the patterning process by the laser patterning apparatus of the processing object according to an embodiment of the present invention may be completed (S7). Of course, the meaning of the completion (S7) means the end of one cycle of the patterning, so that the patterning-completed 3D processing object may be unloaded and new sample 1 which is the 3D processing object may be loaded. However, the processing step S6 is merely laser patterned on the sample 1, which is a three-dimensional processing object, based on the z-axis processing depth data input in the z-axis primary setting step S4, and only performing the two-dimensional position information generation step S3 and the pattern and position information matching step S5 does not go through the actual three-dimensional profiling process, so it cannot be determined that they are processed to the desired processing width, the pattern spacing, and the desired z-axis processing depth.

Therefore, the laser patterning method using the laser patterning apparatus of the processing object according to the embodiment of the present invention may further include the z-axis secondary setting step S8.

As illustrated in FIG. 5, the z-axis secondary setting step S8 is a step of determining the z-axis adjustment amount, which may be performed during the processing step S6 or included in the processing step S6.

More specifically, as illustrated in FIG. 5, the z-axis secondary setting step S8 proceeds through the laser irradiation processing process S8-1 by the execution of the processing step S6, and then goes through the processing shape analysis process S8-2. The processing shape analysis process S8-2 is a process of determining whether the processing width, the pattern spacing, and the z-axis processing depth of the pre-determined pattern are reached as a result of the processing shape analysis of the sample 1, which is the processing object.

As illustrated in FIG. 5, the processing shape analysis process S8-2 may set a new z-axis processing height for matching the z-axis height information of the sample 1, which is the three-dimensional processing object, without completing the processing step S6 if it is determined that the processing line width, the pattern spacing, and the z-axis processing depth of the pre-determined pattern are not reached as a result of matching the three-dimensional processing pattern data input by the three-dimensional processing pattern input step S1-2 based on the two-dimensional shape information obtained from the sample 1, which is the three-dimensional processing object, using the CCD and CMOS camera (not shown).

Here, the processing shape analysis process S8-2 may be performed using one of an optical coherence tomography (OCT), a laser interferometer, a confocal microscope, and a two-photon microscope.

The reprocessing process S8-4 for laser irradiation processing according to the new z-axis processing height given by the z-axis correction process is performed, the processing shape analysis process S8-2 is performed again after the reprocessing process S8-4, and the repetition of the process is terminated only when the processing line width, the pattern spacing, and the z-axis processing depth of the pre-determined pattern are reached.

The calculation required to perform the series of steps and processes described above may be performed through the controller. The calculation includes all calculations required to correct and adjust the processing position and the tilting of the sample 1, which is the three-dimensional processing object, based on the input information which includes the position, arranging status, tilting, 3D outline information and the central point information of the sample 1, which is the three-dimensional processing object

As mentioned above, the z-axis processing depth is already set by the above-described z-axis correction process in the new sample 1, which is loaded again after the patterning completion of the sample 1, which is the three-dimensional processing object, need not go through the z-axis secondary setting step S8, and it is sufficient to perform the patterning process to the z-axis processing depth set in the z-axis primary setting step S4.

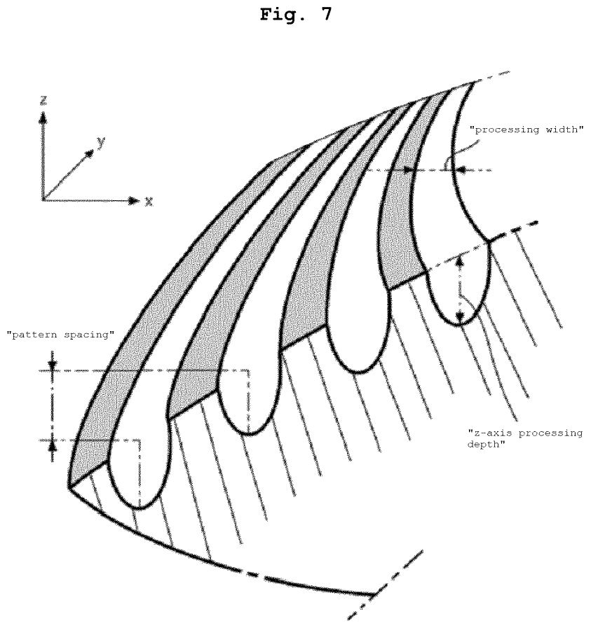

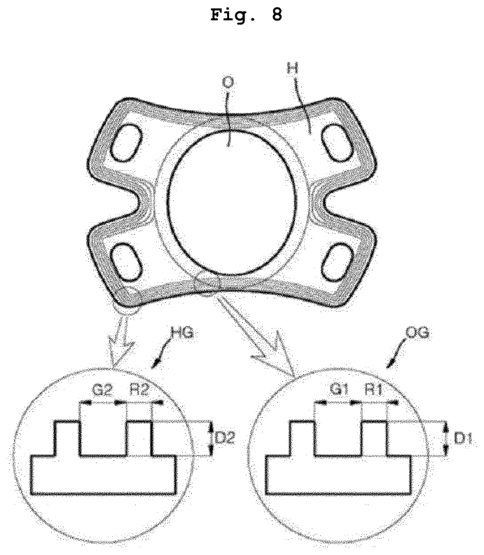

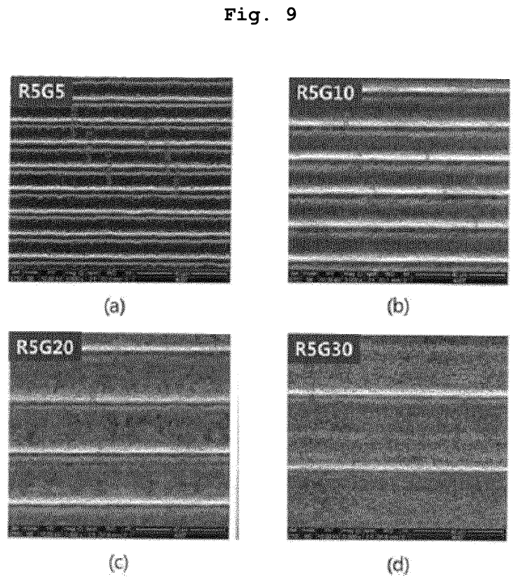

FIG. 6 is a view illustrating an exemplary three-dimensional object according to an embodiment of the present invention, FIG. 7 is an enlarged cross-sectional view of the a-a line of FIG. 6, and FIGS. 8 and 9 are views illustrating pattern shapes according to an embodiment of the present invention.

As an example of the present invention, the three-dimensional object may be an intraocular lens. As illustrated in FIG. 8, the optical part O and the haptic part H may be included. The three-dimensional processing object may include a curved surface of the surface, for example, in the case of an intraocular lens, the optic portion O may have a curved surface. In addition, the processing of the pattern may be formed at least on the optic portion O, and preferably, may be formed near the edge of the optic portion O, which is a circular shape. More precisely, in the case where the three-dimensional processing object is an intraocular lens, the pattern may not be located on the light path into the cornea.

The pattern processed in the intraocular lens may be formed on the curved surface as illustrated in FIG. 7, and each pattern may form a predetermined height difference three-dimensionally, and even if spacing and depth of each pattern are formed the same, the pattern width, the pattern spacing, and the depth of the pattern on the image measured by the two-dimensional plane on the x-y coordinate appears differently according to the height difference because the pattern is formed on the curved surface, therefore, the measured two-dimensional shape information should be used as important information when matching the pattern and the position information.

Referring to FIG. 6, the intraocular lens may be provided as a sample 1 of the processing object, in which a pair of haptic portions O extending from one optic portion O is formed. The pattern formed using the laser patterning apparatus of the processing object according to the embodiment of the present invention may be formed in each region on the haptic portion H and the optic portion O, or may be formed across the two regions. In the following example, the pattern formed across the two regions is described as an example, however it may be applied the same that the features of each patterns formed on the optic portion O and the haptic portion H, for example the information (shape, depth, spacing, and width) of the pattern may be different.

For example, referring to FIGS. 8 and 9, the processing object 1 may be formed in the shape, the depth D1, D2, the spacing R1, R2, and the width G1, G2 of the pattern formed in the intraocular lens differently. That is, the pattern is continuously extended to the haptic portion H and the optic portion O, but the information of the pattern may be formed differently according to the position formed. For example, the spacing R; R1, R2 between the patterns may be determined in the range of 1 to 15 micrometers, and the width G; G1, G2 of the pattern may be determined in the range of 1 to 50 micrometers. In addition, the nano surface internal to the width of the pattern may be determined in the range of 1 to 800 nanometers. Therefore, the pattern may be a composite pattern including a micrometer-level width and a nanometer-level internal surface. Meanwhile, the information of the pattern, that is, the shape, depth, spacing, and width of the pattern, may be determined differently according to the kind of cells moving on the pattern. The spacing between the patterns or the width of the pattern is described above is an example in case the three-dimensional processing object is an intraocular lens and the cells moving on the intraocular lens formed pattern are epithelial cells.

Further, when the patterns including the same spacing R and the different width G are formed, the width G1 of pattern formed on the optic portion O may be smaller than the width G2 of the pattern formed in the haptic portion.

FIG. 10 is a drawing illustrating the processing object after completion of pattering in case the three-dimensional processing object is an intraocular lens.

Referring to FIG. 10, a patterned intraocular lens 1 may be formed through the processing method described above with reference to FIGS. 4 and 5 on the three-dimensional transparent processing object 1. The center of the optic part O may be formed without pattern so that light can pass through without interference with the pattern, and the pattern may be processed on the haptic part H except the optic part o and the peripheral periphery of the optic part O. In the drawing, states of the first pattern 5 and the second pattern 6 may be seen by enlarging at a magnification of 91, 500, and 1000 times, respectively.

Meanwhile, a predetermined structure may be provided in the above-described micro-sized pattern. Here, the predetermined structure may be a nano-sized structure and may be a structure including a nano-sized processing protrusion. In addition, it is possible that the size of the micro-sized pattern and the predetermined structure in the micro-sized pattern may be designed/formed differently depending on the type of cells and the function of the pattern. That is, various cells are existed in the living body, and the micro-sized pattern and the predetermined structure in the micro-sized pattern may be formed differently depending on the type. Furthermore, the pattern size and the predetermined structure in the micro-sized pattern may be formed differently depending on the function of the pattern, such as inhibiting the movement of the cell or activating the movement of the cell.

The processing protrusion may be formed by outputting a processing laser with a size corresponding to the predetermined structure and processing the sample 1, which is the processing object. The processing protrusions may include a side processing protrusion and a bottom processing protrusion. The shape and size of the processing protrusions may be adjusted by a laser processing parameter such as laser output, a laser pulse overlap rate, and an overlap rate between the patterns. Hereinafter, disclosures are explained in case the three-dimensional processing object is an intraocular lens. In addition, in the following description, epithelial cells are described as examples of cells, and are not limited thereto. In fact, in various three-dimensional processing objects, not only epithelial cells, but also various cells in the human body, such as bone cells and inflammatory cells may be controlled in mobility by forming the patterns.

When the three-dimensional processing object is an intraocular lens and the control target cell is epithelial cells, the depth of peripheral portion of the processing protrusion (depth between adjacent processing protrusions) may be formed in a range of 0.1 μm to 30 μm. When the depth is formed to be less than 0.1 μm, the predetermined structure may be difficult to function in order to decrease the mobility of the epithelial cells. In addition, the size of 30 μm, which is the upper limit of the depth, is an example of a size corresponding to the depth of convex part or the height of the concave part, and the upper limit of the predetermined structure provided in the pattern to suppress the posterior or secondary cataract exemplarily.

In addition, the gap between the processing protrusions may be formed in a range of 0.1 μm to 10 μm. If the gap between processing protrusions is formed to be less than 0.1 μm, the epithelial cells causing the posterior cataract may be moved depending the side portion as the area of the side portion being widened, so that it is difficult to expect to suppress the cellular mobility by the processing protrusion. In addition, if the gap between processing protrusions is formed to be over 10 μm, the number of the processing protrusions is reduced, thus it is difficult to expect the cellular mobility may be suppressed by the processing protrusions.

In addition, the width of the processing protrusions may be formed in a range of 0.1 μm to 10 μm. When the width of the processing protrusion is less than 0.1 μm or more than 10 μm, it is difficult to achieve the function for suppressing the mobility of the cell by the processing protrusion.

The predetermined structure may be a boundary portion. Here, the boundary portion may mean a structure that controls the speed and direction in the mobility of the cell moving in the pattern. That is, it means a structure that can increase or decrease the mobility of the cell.

A structure that is narrower from the front side to the rear side based on the movement direction of the cell may be formed. Specifically, in the entire width, one or more extracted portion may be formed in which cross-sectional area of the passage through which the cell moves is reduced to ½ or ⅓, and the movement speed of the cell may be increased. Such a structure may be provided in some sections of the above-described pattern. Here, a portion having a narrow passage cross-sectional area and a wide portion of the cell may be provided in the front and rear side based on the movement direction by the predetermined structure, and may be provided in the opposite direction. That is, the increase or decrease in cell mobility may be controlled according to the conditions required.

The width of the pattern in the state where the cell mobility is highest and the width of the state where the cell mobility is lowest may be formed in the predetermined section by adjusting the laser processing parameter, and the cell mobility may be controlled.

For example, the pattern having the width in the state where the cell mobility is lowest may be applied to the section for lowering the movement speed, and the width in the state where the mobility is high, which is narrower than that, may be provided in the section for promoting the mobility. As an example, the pattern adjacent to the optical portion O formed having the width in the state where the mobility is high.

Further, the boundary portion, which may be the above-described structure, may be a structure that blocks the movement of the cell in one direction in the pattern and bypasses the movement in the other direction (for example, the opposite direction). Such a structure of the boundary portion may function as a structure that suppresses the movement when the cell moves along the movement direction.

Although the representative embodiments of the present invention have been described in detail above, those skilled in the art will understand that various modifications may be made without departing from the scope of the present invention. Therefore, the scope of the present invention should not be limited to the described embodiments but should be determined by equivalents to the claims as well as the claims described below.

1. A laser patterning method of a processing object, the method comprising:
loading a three-dimensional processing object into a laser patterning apparatus, first setting a pre-determined z-axis processing height,
matching two-dimensional position information generated by measuring a shape of the processing object in two dimensions with three-dimensional processing pattern data provided for laser patterning processing of the processing object,
performing processing, and second setting a z-axis processing depth until the z-axis processing height is reached based on the three-dimensional pattern processing pattern data through processing shape analysis of the processing object patterned by the processing.

2. The laser patterning method of claim 1, wherein the two-dimensional position information is acquired from the two-dimensional inspection of the processing object using a camera equipped with CCD or CMOS.

3. The laser patterning method of claim 1, wherein the matching of the two-dimensional position information and the three-dimensional processing pattern data is performed after calculating center point information through the outer contour recognition or area calculation of the processing object acquired through the generation of the two-dimensional position information.

4. The laser patterning method of claim 1, wherein the matching of the 2D position information and the 3D processing pattern data is performed after calculating the center position and tilt information of the processing object through the outer contour recognition or area calculation of the processing object acquired through the generation of the two-dimensional position information.

5. The laser patterning method of claim 1, wherein the pre-determined z-axis processing height is determined by any one of the z-axis according to the processing stage where the processing object is loaded and the z-axis according to the beam regulator of the laser patterning apparatus patterning the processing object.

6. The laser patterning method of claim 1, wherein the processing shape analysis of the processing object is performed by measuring any one of the processing widths and the spacing of the pattern processed to the processing object through one of an optical coherence tomography (OCT), a laser interferometer, a confocal microscope, and a two-photon microscope.

7. A laser patterning method of a processing object, comprising:
loading a three-dimensional processing object into a laser patterning apparatus;
inputting three-dimensional processing pattern data for laser patterning processing of the processing object;
generating two-dimensional position information by measuring the shape of the processing object two-dimensionally;
setting a pre-determined z-axis processing height of the processing object to be processed through the laser patterning apparatus; and
matching the three-dimensional processing pattern data with the two-dimensional position information.

8. The laser patterning method of claim 6, further comprising: after the pattern and position information matching step, performing processing at the z-axis processing height set in the z-axis primary setting step.

9. The laser patterning method of claim 7, wherein the processing step includes a z-axis secondary setting step of analyzing the object to be processed in two dimensions according to the z-axis primary setting step and repeating processing by assigning a new z-axis processing height until the z-axis processing height is reached.

10. The laser patterning method of claim 9, wherein the secondary setting of the z-axis comprises: a processing shape analysis process which two-dimensionally analyzes the pattern processing shape of the object to be processed using one of an optical coherence tomography (OCT), a laser interferometer, a confocal microscope, and a two-photon microscope; and
a z-axis correction process of assigning a new z-axis processing height when one of the pattern processing width, pattern spacing, and pattern depth of the processing object analyzed by the processing shape analysis process does not reach one of the pattern width, pattern spacing, and pattern spacing information on the three-dimensional processing pattern data.

11. The laser patterning method of claim 10, wherein the processing shape analysis process is performed by measuring any one of the processed line widths and the spacing of the pattern processed on the surface of the object.

12. The method of claim 7, further comprising a quality analysis and reporting step of analyzing and reporting any one of a shape of a pattern, a width of a pattern, a depth of a pattern, a spacing between patterns, a wavelength and an output of a laser beam, a pulse width, a shape of a beam, a scanning speed, and a spot size for the processing object to which the pattern is processed.

13-14. (canceled)
